SEQUENCE LISTING (19 sequences)

SEQ ID NO: 1 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 1445 aa, Artificial sequence
  scFVIII amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 3 - protein, 33 aa, Homo sapiens
PRFQDSSSSKAPPPSLPSPSRLPGPSDTPILPQ

SEQ ID NO: 4 - protein, 227 aa, Artificial sequence
  vFcgamma1 amino acid sequence
DKTHTCPPCPAPEVAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 223 aa, Artificial sequence
  vFcgamma2-1 amino acid sequence
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 6 - protein, 223 aa, Artificial sequence
  vFcgamma2-2 amino acid sequence
VECPPCPAPPVAGPSVFLFPPKPKDQLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPGK

SEQ ID NO: 7 - protein, 223 aa, Artificial sequence
  vFcgamma2-3 amino acid sequence
VECPPCPAPPVAGPSVFLFPPKPKDQLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPGK

SEQ ID NO: 8 - protein, 229 aa, Artificial sequence
  vFcgamma4 amino acid sequence
ESKYGPPCPPCPAPEFAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 1723 aa, Artificial sequence
  amino acid sequence of scFVIII fusion protein
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYGSGGGGSGGGGSGGGGSGGGGSGGGGSSSSSKAPPPSLPSPSRLPGPSDTPILPQVECPPCPAPPVAGPSVFLFPPKPKDQLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPGK

SEQ ID NO: 10 - DNA, 5169 bp, Artificial sequence
  nucleotide sequence of scFVIII fusion protein
gccaccaggaggtactacctcggagccgtcgagctcagctgggattatatgcagagcgacctcggagaactccctgtcgatgccaggtttcccccccgggtgcccaagtccttcccctttaacacctccgtcgtctacaagaagaccctgttcgtcgagttcacagaccatctgttcaatattgctaaacccaggcccccctggatgggactgctgggccccacaatccaagccgaggtgtatgataccgtggtgatcaccctcaagaatatggcttcccatcccgtctccctgcacgccgtcggcgtgtcctactggaaggcctccgaaggcgccgagtacgatgatcagacaagccagcgggagaaggaggatgataaggtctttcccggaggttctcacacctatgtctggcaggtgctgaaagagaacggccccatggctagcgaccccctgtgcctcacctattcctacctcagccatgtggacctggtgaaggatctgaactccggcctgattggcgccctcctggtctgcagggagggaagcctggccaaagaaaagacccagaccctgcataaattcatcctcctgtttgccgtcttcgatgagggcaagtcctggcactccgaaacaaagaactccctgatgcaggacagggacgctgcctccgccagggcctggcccaagatgcacaccgtcaacggatacgtgaataggtccctccccggcctgattggatgccatcggaagtccgtgtattggcacgtgatcggcatgggaaccacacccgaagtgcactccatcttcctggaaggacatacatttctcgtccggaaccaccggcaggcttccctggaaatctcccctatcaccttcctgaccgctcagacactgctgatggatctgggccagttcctgctgttctgccacatttcctcccaccagcacgacggcatggaggcttatgtcaaggtggatagctgccccgaggaaccccagctgcggatgaaaaacaatgaggaagccgaggactacgacgatgacctcaccgatagcgagatggacgtggtcaggttcgacgacgataactccccttccttcatccagatccggagcgtggccaagaagcaccccaaaacctgggtgcactacatcgccgctgaggaggaagactgggactatgctcctctcgtgctggcccccgatgacaggtcctacaaaagccagtatctgaacaatggaccccagcggattggaaggaagtataagaaagtccggtttatggcttacaccgacgagacctttaaaacccgggaagccatccagcacgagagcggcatcctcggccctctcctgtacggagaggtcggagacacactcctcatcatttttaagaaccaggcctcccggccctacaacatctatcctcatggcatcaccgatgtgaggcctctgtacagcaggaggctgcccaaaggagtcaagcacctgaaagatttccctatcctccccggagagatcttcaagtacaaatggaccgtcaccgtggaggacggccccacaaagtccgatccccggtgcctgacccggtactattccagctttgtcaacatggagcgggacctcgccagcggcctgatcggccctctgctcatctgctataaggagtccgtcgatcagaggggaaaccagatcatgtccgataagcggaatgtgatcctgttttccgtgtttgacgaaaaccggtcctggtatctgaccgagaacatccagcggtttctgcctaaccccgctggcgtgcaactggaagatcctgagttccaggcttccaatatcatgcacagcatcaacggctacgtcttcgacagcctgcagctgtccgtctgcctgcacgaagtcgcctactggtacattctcagcattggcgcccagacagacttcctgtccgtctttttctccggctataccttcaaacataagatggtctacgaggataccctcacactgttccctttcagcggcgaaaccgtgtttatgagcatggaaaatcccggcctgtggattttaggatgccacaactccgatttcaggaataggggcatgacagctctcctcaaagtgtcctcctgcgacaagaacacaggcgactactacgaggacagctatgaggatatttccgcctacctgctcagcaagaataatgccatcgagcctaggtccttctcccaaaactcccggcatcccagcacccggcagaagcagttcaatgccaccacaattcctgaaaatagaacaaccctgcagtccgaccaggaggaaatcgactacgatgacaccatctccgtcgagatgaagaaggaagacttcgacatctatgacgaggacgaaaaccagagccctcggtcctttcagaaaaaaacacggcattactttatcgctgccgtcgaacggctgtgggactacggcatgagctcctccccccatgtgctccggaatcgggctcaatccggctccgtgcctcagtttaagaaggtggtgtttcaagagttcaccgacggcagctttacacagcccctgtacaggggagagctgaacgagcacctgggactgctcggaccttacattcgggccgaagtcgaggataacatcatggtgacctttaggaatcaggcttcccggccttactccttctactccagcctcatctcctacgaagaagaccaacggcagggcgccgagcctcggaagaatttcgtgaagcccaatgaaaccaaaacatacttttggaaagtccagcaccacatggcccctaccaaggatgagttcgactgcaaagcctgggcctatttctccgatgtggatctggagaaggacgtccattccggactgatcggccccctcctcgtctgccacaccaacaccctcaatcctgcccacggcaggcaggtgacagtccaggagttcgccctgtttttcaccatctttgacgagaccaagtcctggtactttaccgagaatatggaaaggaattgcagggccccctgcaacattcagatggaggaccccaccttcaaagagaactaccggtttcatgccattaatggatacatcatggacacactgcccggcctggtgatggctcaggaccaacggattcggtggtacctgctgagcatgggctccaacgaaaatatccacagcatccactttagcggccacgtgtttaccgtgcggaagaaggaggagtataaaatggccctctacaacctctatcccggcgtgttcgaaacagtcgagatgctccctagcaaggccggaatctggagggtggaatgcctgattggcgaacatctgcacgccggaatgagcaccctcttcctggtctacagcaacaaatgtcagacccctctgggcatggcttccggccacatcagggactttcagattaccgctagcggacaatatggccagtgggcccctaaactcgctaggctgcactacagcggaagcatcaacgcctggtccacaaaggaacctttctcctggatcaaggtggatctcctcgcccctatgatcatccatggcatcaagacccaaggcgctaggcagaagtttagctccctgtacatcagccagtttatcatcatgtactccctcgacggcaagaagtggcagacataccggggaaactccacaggcaccctgatggtgttctttggcaacgtggacagcagcggaattaagcataatattttcaaccctcctatcatcgccaggtacatccggctgcaccctacacactacagcatcaggagcaccctgaggatggagctgatgggctgcgatctgaatagctgctccatgcccctcggcatggagagcaaggctatttccgacgcccaaattaccgccagctcctatttcaccaacatgttcgccacatggagccctagcaaagctcggctccatctccagggcaggtccaacgcctggcggcctcaggtgaataatcccaaagaatggctgcaggtcgacttccagaagacaatgaaagtgaccggcgtgaccacccagggagtcaaaagcctcctcaccagcatgtacgtcaaagagttcctgatcagctcctcccaggacggacatcaatggacactcttttttcagaacggcaaggtgaaggtgttccagggcaaccaggattcctttacccctgtggtcaactccttagatcctcccctcctgacacggtacctgcggattcaccctcagtcctgggtgcatcagatcgctctgaggatggaagtgctcggctgcgaagcccaagacctgtacggatccggtggcggtggctccggtggaggcggaagcggcggtggaggatcaggcggtggaggtagcggcggaggcggtagctccagctctagtaaagctccccctccttccctgccctcaccctcaagactgcctggaccttccgacactcccatcctgccacaggtggagtgccctccatgtccagcaccccctgtcgcaggtccatctgtgttcctgtttccacccaagcctaaagaccagctgatgatctcccgcaccccagaagtcacctgtgtggtcgtggatgtgagccatgaagaccccgaggtccagttcaattggtacgtggatggcgtcgaggtgcacaacgctaagacaaaacctagagaagagcagttcaactctacctttcgcgtcgtgagtgtgctgacagtcgtgcaccaggactggctgaatggcaaggagtataagtgcaaagtgagcaacaaaggactgcctgcctcaatcgaaaagactatttccaagaccaaaggacagccaagagagccccaggtgtacaccctgcctccaagccgcgaagagatgactaaaaatcaggtctctctgacctgtctggtgaaggggttttatcctagtgatatcgccgtggaatgggagtcaaacggtcagccagagaacaattacaagaccacaccccctatgctggacagcgatgggtctttctttctgtatagcaaactgacagtggacaagtctcggtggcagcagggtaacgtcttctcttgcagtgtgctgcacgaagcactgcacaatcattacacccagaagtcactgtcactgagcccaggaaaa

SEQ ID NO: 11 - protein, 1716 aa, Artificial sequence
  amino acid sequence of DS-F8 fusion protein
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYGSGGGGSGGGGSGGGGSGGGGSGGGGSSSSSKAPPPSLPSPSRLPGPSDTPILPQVECPPCPAPPVAGPSVFLFPPKPKDQLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPGK

SEQ ID NO: 12 - protein, 28 aa, Artificial sequence
  Artificially synthesized peptide linker
SSSSRAPPPSLPSPSRLPGPSDTPILPQ

SEQ ID NO: 13 - protein, 10 aa, Artificial sequence
  Artificially synthesized peptide linker
SSSSRAPPPS

SEQ ID NO: 14 - protein, 14 aa, Artificial sequence
  Artificially synthesized peptide linker
SRLPGPSDTPILPQ

SEQ ID NO: 15 - protein, 16 aa, Artificial sequence
  Artificially synthesized peptide linker
GSGGGSGGGGSGGGGS

SEQ ID NO: 16 - protein, 28 aa, Artificial sequence
  Artificially synthesized peptide linker
GSGGGGGSGGGGSGGGGSGGGGSGGGGS

SEQ ID NO: 17 - protein, 20 aa, Artificial sequence
  Artificially synthesized peptide linker
GGGGSGGGGSGGGGSGGGGS

SEQ ID NO: 18 - protein, 37 aa, Artificial sequence
  Artificially synthesized peptide linker
GSGGGGSGGGGSGGGGSGGGGSGGGGSGGGGSGGGGS

SEQ ID NO: 19 - protein, 19 aa, Artificial sequence
  Artificially synthesized peptide linker
GGGSGGGSGGGSGGSGGGS

(19) United States
(12) Patent Application Publication
(10) Pub. No.: US 2022/0033476 A1
(43) Pub. Date: Feb. 3, 2022

(54) FUSION PROTEIN OF MUTATED SINGLE-CHAIN HUMAN COAGULATION FACTOR VIII, PREPARATION METHOD THEREFOR, AND USE THEREOF

(71) Applicants: AMPSOURCE BIOPHARMA SHANGHAI INC., Shanghai (CN); PHARMAB, INC., Shanghai (CN)

(72) Inventors: Yongjuan Gao, Shanghai (CN); Shixiang Jia, Shanghai (CN); Yuncheng Zheng, Shanghai (CN); Yingying Jin, Shanghai (CN); Zhu Wang, Shanghai (CN); Zhao Dong, Shanghai (CN); Si Chen, Shanghai (CN); Bill Nai-chau Sun, Shanghai (CN); Qiang Li, Shanghai (CN)

(21) Appl. No.: 17/280,343

(22) PCT Filed: Sep. 24, 2019
(86) PCT No.: PCT/CN2019/107432

§ 371 Date: Mar. 26, 2021

(30) Foreign Application Priority Data
Sep. 26, 2018 (CN) 201811123918.X

(51) Int. Cl. C07K 14/755; C07K 14/59

(52) U.S. Cl. CPC C07K 14/755; C07K 14/59; A61K 38/00; C07K 2319/30; C07K 2319/31

(57) ABSTRACT

Disclosed is a fusion protein of a mutated recombinant single-chain human coagulation factor VIII (FVIII), a preparation method therefor, and a use thereof. The fusion protein sequentially comprises, from an N-terminus to a C-terminus, a mutated single-chain human FVIII having a partially deleted B-domain, a flexible peptide linker, at least one rigid unit of a carboxyl-terminal peptide of a human chorionic gonadotropin beta subunit, and a half-life prolonging moiety (preferably an IgG Fc variant). The fusion protein has a similar biological activity to a recombinant FVIII, a prolonged active half life in vivo, and better stability in vitro and in vivo, and thus improves the pharmacokinetics and efficacy of the fusion protein.

### TECHNICAL FIELD

The present disclosure relates to the field of fusion proteins and, in particular, to a fusion protein of a mutant human coagulation factor VIII (FVIII), a preparation method thereof, and use thereof, especially use for treating a variety of coagulation-related diseases.

### BACKGROUND

Coagulation factor VIII (FVIII), also referred to as an anti-hemophilic factor, plays an important role in an endogenous coagulation system. The results of numerous studies on FVIII in molecular genetics show that deficiency of FVIII in sex chromosome X-linked genes results in hemophilia A. According to statistics, the prevalence of hemophilia A in males is 1/5000 and accounts for more than 80% of the total number of hemophilia patients. At present, a common treatment for hemophilia A is replacement therapy, that is, the supplement of FVIII that hemophilia patients lack.

Mature FVIII consists of light and heavy chains with a molecular weight of approximately 280 kDa and has domains A1-A2-B-A3-C1-C2. In cells, the proteolysis of residue Arg-1648 in the B domain produces a heavy chain (A1-A2-B) with different sizes ranging from 90 to 200 kDa and a light chain (domains A3-C1-C2) of 80 kDa. The heavy chain and the light chain bind to form a heterodimer via divalent metal ion-dependent links. In plasma, the dimer consisting of a heavy chain and a light chain then binds to von Willebrand Factor (vWF) with high affinity to be protected from being degraded prior to maturation.

The half-life of non-activated FVIII that binds to vWF is approximately 12 hours in plasma. FVIII is activated through hydrolytic cleavage of Arg372 and Arg740 in the heavy chain and Arg1689 in the light chain by activated coagulation factor FX (FXa) and thrombin FII (FIIa), so that vWF factor is released and activated FVIII dimers (FVIIIa) are generated. The FVIIIa forms a tight complex with activated coagulation factor FIX (FIXa) and FX on the surface of phospholipid in the presence of Ca²⁺. FX is then activated by FIXa. The activated FX is dissociated from the complex and converts prothrombin to thrombin, where thrombin can directly convert fibrinogen to fibrin. As a cofactor in the coagulation system, FVIII can increase the catalytic efficiency of FIXa for activating FX by several orders of magnitude.

Due to a complex structure and a large molecular weight of FVIII protein, FVIII is very unstable and easy to deactivate in the processes such as plasma collection and recombinant purification and preparation. FVIII takes effect within a relatively short period, especially in an aqueous solution. Factors such as storage temperature, inorganic salt ions, trace proteases, and other proteins involved in coagulation (especially vWF and albumin) all affect the stability of FVIII molecules. During injection administration after the reconstitution of FVIII lyophilisates, safety and sterility must be guaranteed, and the infusion must be completed within specified time which is generally no more than 4 hours. On the other hand, violent oscillations when the FVIII lyophilisates are reconstituted will also result in damages to the structure of proteins, which deactivates FVIII. Therefore, the development of FVIII molecules with better stability can improve the flexibility of FVIII products in clinical applications and is essential to greatly improve the medication safety and quality of life of patients.

The B domain of FVIII contains 18 N-glycosylation sites, has no known function related to coagulation, and is not homologous to other proteins. FVIII molecules with the B domain deleted still have good procoagulant activity. Eaton et al. disclose that an FVIII molecule that lacks 766 amino acids (797 to 1562) in the central B domain retains biological activity; in addition, FVIII with the B domain deleted is expressed in higher amounts than full-length molecules in mammal cells and exhibits a faster and higher activation rate than the full-length molecules (Eaton et al., Biochemistry, 1986, 25: 8343-8347). Other studies also show that deletion of major portion of the B domain (Ser743 to GIn1638) have no effect on the functional activity of FVIII, but significantly decrease the molecular weight (by 38%), and increase an expression level of FVIII in eukaryotic cells (Peters R T et al., J Thromb Haemost, 2013, 11(1): 132-141; Sandberg H et al., Semin Hematol, 2001, 38: 4-12).

Afstyla is a new recombinant single-chain human FVIII product and is the only single-chain coagulation factor product with a light chain and a heavy chain fused together, which is currently approved for the treatment of hemophilia A. Afstyla has higher molecular stability and longer duration of efficacy due to its strong affinity with von Willebrand Factor (vWF). In clinical studies, Afstyla demonstrates excellent hemostasis and bleeding prophylaxis effects in both a prophylaxis and an on-demand treatment, with relatively low dosage and very good safety in both administration schemes. Compared with a standard care drug Octocog alfa, Afstyla is more stable in protein configuration and has relatively durable efficacy, but still needs to be injected 2-3 times per week.

To extend the in vivo functional half-life of FVIII, in the existing art, FVIII is linked to a half-life extending moiety such as PEG, human serum albumin (HSA), transferrin, or IgG Fc. Currently, Novo Nordisk (N8-GP), Bayer (BAY94-9027), and Baxter (BAX 855) have developed long-lasting PEGylated FVIII products which have entered the stage of clinical studies. However, an additional step of chemically conjugating PEG to FVIII is added to a protein preparation process, reducing a final yield and increasing a preparation cost. On the other hand, data of pharmacokinetic studies shows that PEGylated FVIII has not achieved a significantly prolonged half-life (Tiede A et al., J Thromb Haemost, 2013, 11: 670-678; Turecek P L et al., Hamostaseologie, 2012, 32 Suppl 1: S29-38). WO2013106789 has disclosed a chimeric polypeptide (FVIIIFc) containing an FVIII moiety and an Fc moiety, where the terminal half-life of the chimeric FVIII polypeptide is extended to twice longer than that of rFVIII. In the prophylaxis, it is administered twice per week to maintain the level of FVIII activity at 1-3%.

CTP is a short peptide derived from the carboxyl terminus of human chorionic gonadotropin (hCG) β-subunit and contains multiple O-glycosylation sites. This negatively charged and highly sialylated peptide is covalently linked to the C-terminus of other proteins so as to be resistant to renal clearance, thereby extending the half-life of a target protein to which the CTP is linked in vivo. Some patent documents disclose that the CTP may be used as a half-life extending moiety included in a fusion protein. The present disclosure creatively uses a CTP polypeptide with multiple O-glycosylation sites as a part of a linker peptide for linking single-chain FVIII and an Fc fragment instead of being linked to the C-terminus to function as a fusion ligand. Since it has natural glycosylation sites, the CTP polypeptide can not only further prolong the half-life of the fusion protein and improve bioavailability but also cooperate with a conventional flexible GS flexible linker peptide to form a stable stereo conformation, thereby prompting single-chain FVIII and the Fc fragment to be independently folded to form a correct three-dimensional conformation, greatly reducing the steric hindrance effect of the fusion ligand Fc on single-chain FVIII, and enabling single-chain FVIII to maintain relatively high biological activity. In addition, the protective effect of CTP glycosyl side chains can reduce the susceptibility of the linker peptide to proteases.

In summary, although currently many studies provide effective schemes for preparing recombinant FVIII proteins, the heterologous number, structures and unstable thrombin activation of FVIII derivatives compared with full-length FVIII are still a serious problem. In addition, since a variety of B domain deleted FVIII are expressed as fusion proteins, the non-natural amino acid sequences may cause new immunogenicity when administrated. The stability of FVIII molecules is also critical for storage and clinical applications. In this case, it is desirable to develop activated, stable, and safe FVIII derivatives that can have the same thrombin activity, an increased yield, and a longer half-life so that the prophylaxis can be maintained by reducing the frequency of injections, helping to improve treatment effects.

### SUMMARY

The present disclosure provides an Fc fusion protein of a recombinant mutant single-chain coagulation factor VIII, which has an extended activity half-life in vivo and biological activity similar to that of recombinant FVIII. In addition, the present disclosure provides a method for efficiently and stably expressing the fusion protein. The fusion protein expressed by the method has the advantages of high yield, good stability in preparation and storage processes, and biological activity similar to that of recombinant FVIII on the market. The inventors of the present application have surprisingly found that the constructed recombinant mutant single-chain FVIII fusion protein has substantially enhanced stability, the fusion protein can avoid being cleaved by proteases in cells, and the obtained fusion protein exhibits better stability after being purified and good bioavailability when subcutaneously administered.

In a first aspect of the present disclosure, the present disclosure relates to a recombinant mutant single-chain coagulation factor VIII fusion protein. The fusion protein includes, sequentially from the N-terminus to the C-terminus, a single-chain human FVIII with a partially deleted B domain (scFVIII), a flexible peptide linker (L), at least one carboxyl-terminal peptide rigid unit of human chorionic gonadotropin β-subunit (hereinafter referred to as “CTP rigid unit” and expressed as (CTP)ₙ, wherein n is preferably 1, 2, 3, 4, or 5), and a half-life extending moiety (such as an immunoglobulin Fc fragment, albumin, transferrin, or PEG, preferably a human IgG Fc variant (expressed as vFc)). In some preferred embodiments of the present disclosure, the fusion protein is expressed as scFVIII-L-CTP-vFc.

The scFVIII lacks amino acids 765 to 1651 compared with the amino acid sequence of full-length human wild-type FVIII as shown in SEQ ID NO: 1. Specifically, the scFVIII has an amino acid sequence as shown in SEQ ID NO: 2.

The flexible peptide linker is preferably non-immunogenic, and produces a sufficient spatial distance between scFVIII and Fc to minimize steric hindrance therebetween. Preferably, used herein is a flexible peptide linker consisting of two or more amino acid residues which are selected from the following amino acids: Gly(G), Ser(S), Ala(A), and Thr(T).

More preferably, the flexible peptide linker includes G and S residues. The length 10 of a linker peptide is very important for the activity of the fusion protein. For the present disclosure, the peptide linker may preferably have an amino acid sequence general formula formed by combining cyclic units (GS)a(GGS)b(GGGS)c(GGGGS)d, wherein a, b, c, and d are integers greater than or equal to 0 and a+b+c+d≥1.

Specifically, in an embodiment of the present disclosure, the peptide linker may preferably include a following sequence:

| | |
|---|---|
| (i) L1: | GSGGGSGGGGSGGGGS; |
| (ii) L2: | GSGGGGSGGGGSGGGGSGGGGSGGGGS; |
| (iii) L3: | GGGGSGGGGSGGGGSGGGGS; |
| (iv) L4: | GSGGGGSGGGGSGGGGSGGGGSGGGGSGGGGSGGGGS; |
| (v) L5: | GGGSGGGSGGGSGGGSGGGS. |

The CTP rigid unit is selected from a full-length sequence consisting of amino acids 113 to 145 at the carboxyl-terminus of human chorionic gonadotropin β-subunit or a fragment thereof. Specifically, the CTP rigid unit includes an amino acid sequence as shown in SEQ ID NO: 3 or a truncated sequence thereof. First, such CTP polypeptide that contains multiple glycosylation sites and naturally exists in human bodies is non-immunogenic. Secondly, compared to the flexible linker peptide that curls irregularly, a CTP rigid linker peptide containing multiple glycosylation sites can form a stable stereo conformation and promote the scFVIII and the Fc fragment to be independently folded to form a correct three-dimensional conformation without affecting their respective biological activity. In addition, the protective effect of CTP glycosyl side chains can reduce the susceptibility of the linker peptide to proteases.

Preferably, the CTP rigid unit includes at least two glycosylation sites. For example, in a preferred embodiment of the present disclosure, the CTP rigid unit includes two glycosylation sites. Exemplarily, the CTP rigid unit includes 10 amino acids at the N-terminus of SEQ ID NO: 3, that is, SSSS*KAPPPS*. Alternatively, the CTP rigid unit includes 14 amino acids at the C-terminus of SEQ ID NO: 3, that is, S*RLPGPS*DTPILPQ. For another example, in another embodiment, the CTP rigid unit includes three glycosylation sites. Exemplarily, the CTP rigid unit includes 16 amino acids at the N-terminus of SEQ ID NO: 3, that is, SSSS*KAPPPS*LPSPS*R. For another example, in some other embodiments, the CTP rigid unit includes four glycosylation sites. Exemplarily, the CTP rigid unit includes 28, 29, 30, 31, 32, or 33 amino acids from position 113, 114, 115, 116, 117, or 118 to position 145 of the human chorionic gonadotropin β-subunit. Specifically, the CTP rigid unit includes 28 amino acids at the N-terminus of SEQ ID NO: 3, that is, SSSS*KAPPPS*LPSPS*RLPGPS*DTPILPQ. Herein, * represents a glycosylation site. Each possibility represents an independent embodiment of the present disclosure.

In some other embodiments, the CTP rigid unit provided by the present disclosure is at least 70% homologous to a native CTP amino acid sequence. In some other embodiments, the CTP rigid unit provided by the present disclosure is at least 80% homologous to a native CTP amino acid sequence. In some other embodiments, the CTP rigid unit provided by the present disclosure is at least 90% homologous to a native CTP amino acid sequence. In some other embodiments, the CTP rigid unit provided by the present disclosure is at least 95% homologous to a native CTP amino acid sequence.

In a specific embodiment of the present disclosure, the CTP rigid unit may preferably include a following sequence:

| | |
|---|---|
| (i) CTP¹: | PRFQDSSSSKAPPPSLPSPSRLPGPSDTPILPQ; |
| (ii) CTP²: | SSSSKAPPPSLPSPSRLPGPSDTPILPQ; |
| (iii) CTP³: | SSSSKAPPPS; |
| (iv) CTP⁴: | SRLPGPSDTPILPQ. |

In some embodiments of the present disclosure, the fusion protein includes one CTP rigid unit described above. In some other embodiments of the present disclosure, the fusion protein includes two or more CTP rigid units described above, preferably 2, 3, 4, or 5 CTP rigid units described above.

The half-life extending moiety is preferably selected from an immunoglobulin IgG, IgM, or IgA Fc fragment, more preferably selected from an Fc fragment of human IgG1, IgG2, IgG3, or IgG4 or a variant thereof. Further, the human IgG Fc variant includes at least one amino acid modification in wild-type human IgG Fc, and the variant has a reduced effector function (ADCC and/or CDC effects) and/or enhanced binding affinity to a neonatal receptor FcRn. Further, the human IgG Fc variant may be selected from the following group:

(i) vFcγ₁: human IgG1 hinge region, CH2 region, and CH3 region containing Leu234Val, Leu235Ala, and Pro331Ser mutations (such as an amino acid sequence as shown in SEQ ID NO: 4);

(ii) vFcγ₂₋₁: human IgG2 hinge region, CH2 region, and CH3 region containing Pro331Ser mutation (such as an amino acid sequence as shown in SEQ ID NO: 5);

(iii) vFcγ₂₋₂: human IgG2 hinge region, CH2 region, and CH3 region containing Thr250Gln and Met428Leu mutations (such as an amino acid sequence as shown in SEQ ID NO: 6);

(iv) vFcγ₂₋₃: human IgG2 hinge region, CH2 region, and CH3 region containing Pro331Ser, Thr250Gln, and Met428Leu mutations (such as an amino acid sequence as shown in SEQ ID NO: 7);

(v) vFcγ₄: human IgG4 hinge region, CH2 region, and CH3 region containing Ser228Pro and Leu235Ala mutations (such as an amino acid sequence as shown in SEQ ID NO: 8).

The IgG Fc variant provided by the present disclosure includes, but is not limited to, the above five variants in (i) to (v) and may also be a combination or superposition of mutation sites in two types of functional variants of IgG isotypes. For example, the variant in (iv) is a new IgG2 Fc combined variant obtained by superposing mutation sites in (ii) and (iii).

The Fc variant (vFc) in the fusion protein of the present disclosure contains the hinge region, CH2 region, and CH3 region of human IgG such as human IgG1, IgG2, or IgG4. The CH2 region contains amino acid mutations at positions 228, 234, 235, and 331 (determined by an EU counting system). It is believed that these amino acid mutations can reduce the effector function of Fc. Human IgG2 does not bind to FcγR but shows very low complement activity. An Fcγ2 variant with Pro331Ser mutation should have lower complement activity than natural Fcγ2 and remains unbound to FcγR. IgG4 Fc is defective in activating complement cascade and its binding affinity to FcγR is about an order of magnitude lower than IgG1. An Fcγ4 variant with Leu235Ala mutation should exhibit a minimum effector function compared with natural Fcγ4. Fcγ1 with Leu234Val, Leu235Ala, and Pro331 Ser mutations also exhibits reduced effector function compared with natural Fcγ1. These Fc variants are all more suitable for the preparation of a FVIII fusion protein than natural human IgG Fc. Meanwhile, amino acid mutations at positions 250 and 428 (determined by an EU numbering system) increase the binding affinity of an Fc region to the neonatal receptor FcRn, further extending the half-life (Paul R et al., J Biol Chem, 2004, 279: 6213-6216). The combination or superposition of the above two types of functional variants gives a new combined variant which not only reduces the effector function but also extends the half-life. The Fc variant of the present disclosure includes, but is not limited to, mutations at the above several sites. Substitutions at other sites may also be introduced so that Fc has the reduced effector function and/or enhanced binding to an FcRn receptor without causing a decrease in the function/activity of the Fc variant or causing an adverse conformational change. For common mutation sites, see Shields R L et al., J Biol Chem, 2001, 276(9): 6591-604.

In a preferred embodiment of the present disclosure, the fusion protein has an amino acid sequence as shown by SEQ ID NO: 9.

In another aspect of the present disclosure, DNA for encoding the fusion protein described above is provided.

In a preferred embodiment of the present disclosure, the DNA for encoding the fusion protein has a sequence as shown in SEQ ID NO: 10.

In another aspect of the present disclosure, a vector is provided. The vector includes the DNA described above.

In another aspect of the present disclosure, a host cell is provided. The host cell includes the vector described above or has been transfected with the vector described above.

In a specific embodiment of the present disclosure, the host cell is a CHO-derived cell strain DXB-11.

In a fifth aspect of the present disclosure, a pharmaceutical composition is provided. The pharmaceutical composition includes a pharmaceutically acceptable carrier, excipient, or diluent and an effective amount of the fusion protein described above.

In another aspect of the present disclosure, a method for preparing or producing the fusion protein from a mammal cell line such as a CHO-derived cell line is provided, including the following steps:

(a) introducing DNA for encoding a fusion protein into a CHO cell to produce a CHO-derived cell line;

(b) screening in step (a) a high-yielding cell strain expressing more than 1 IU/10⁶ cells every 24 hours in a growth medium thereof;

(c) culturing the cell strain screened in step (b) to express the fusion protein;

(d) harvesting a fermentation broth obtained in step (c) and separating and purifying the fusion protein.

Further, the CHO-derived cell line in step (a) is DXB-11.

Further, cell culture in step (c) may be batch culture, perfusion culture, or fed-batch culture.

Further, in the step (d), the fusion protein is purified by four-step chromatography, namely, affinity chromatography, hydrophobic chromatography, anion exchange chromatography, and molecular sieve chromatography. Preferred conditions are further described in the present disclosure in conjunction with Example 5.

In a preferred embodiment of the present disclosure, the fusion protein prepared by the above method has activity of greater than 6000 IU/mg.

In a sixth aspect of the present disclosure, use of the fusion protein for preparing a medicament for the prevention or treatment of a hemorrhagic disease or event due to an FVIII deficiency or a functional deficiency is provided.

Further, the disease includes hemophilia A. The fusion protein of the present disclosure plays a role in controlling or preventing the occurrence of bleeding in the spontaneous bleeding event, surgical prophylaxis, perioperative management, or surgical treatment of a patient with hemophilia A.

The disclosed and/or described fusion protein and the preparation method thereof of the present disclosure have the following advantages:

1. The Fc fragment in the mutant single-chain FVIII fusion protein constructed in the present disclosure is non-lytic. That is, the complement and receptor binding domains of the Fc fragment are mutated to regulate the binding affinity of Fc to corresponding receptors so as to reduce or eliminate ADCC and CDC effects, such that the Fc fragment extends the in vivo half-life of active proteins without producing cytotoxicity.

2. The mutant single-chain FVIII fusion protein provided by the present disclosure includes a rigid CTP polypeptide with multiple glycosyl side chains. Compared to a flexible linker peptide such as (GGGGS)n that curls irregularly, the rigid CTP polypeptide can form a stable stereo conformation. This “separation” role promotes FVIII and the Fc fragment to be independently folded to form the correct three-dimensional conformation without affecting their respective biological activity. The CTP contains multiple O-modified oligosaccharides. The negatively charged and highly sialylated CTP can resist renal clearance and further extend the half-life of the fusion protein. On the other hand, the protective effect of CTP glycosyl side chains can reduce the susceptibility of the linker peptide to proteases so that the fusion protein is not easily degraded in the linker region.

3. The fusion protein of the present disclosure has good in vitro stability during fermentation, purification, and storage. After storage at room temperature i.e., 25° C. for seven days, the activity of the mutant single-chain FVIII fusion protein is significantly higher than that of a medicament of a recombinant double-chain FVIII Fc fusion protein where the protease cleavage sites of the fusion protein between Arg1648 and Glu1649 are not inactivated (encoded by a human wild-type FVIII full-length sequence i.e., SEQ ID NO: 1), and the activity loss is less than 20%.

4. The method for preparing the fusion protein provided by the present disclosure can achieve a high yield. An accumulated yield can reach at least 200 mg/L after incubation in a 300 mL shake flask for 14 days. The process can be scaled up to achieve large-scale industrial production.

### DETAILED DESCRIPTION OF THE DISCLOSURE

Carboxyl-terminal peptide (CTP) of hCG-β CTP is a short peptide derived from the carboxyl terminus of human chorionic gonadotropin (hCG) β-subunit. Four reproduction-related polypeptide hormones comprising follicle-stimulating hormone (FSH), luteinizing hormone (LH), thyroid-stimulating hormone (TSH), and human chorionic gonadotropin (hCG), contain the same α-subunit and their respective specific β-subunits. The in vivo half-life of hCG is significantly longer than those of the other three hormones, mainly due to the carboxyl-terminal peptide (CTP) specific to its β-subunit (Fares F A et al., Proc Natl Acad Sci USA, 1992, 89(10): 4304-4308). A natural CTP contains 37 amino acid residues, four O-glycosylation sites, and a terminal sialic acid residue. The negatively charged and highly sialylated CTP can resist renal clearance and thus extend the circulation half-life in vivo (Fares F A et al., Proc Natl Acad Sci USA, 1992, 89(10): 4304-4308). However, the present disclosure creatively links at least one CTP polypeptide with a flexible linker peptide with an appropriate length, which together serve as a linker peptide for linking FVIII to a half-life extending moiety (such as an immunoglobulin Fc fragment).

The present disclosure has found that the addition of the CTP between FVIII and an Fc variant is equivalent to the addition of a rigid linker peptide. This, on one hand, ensures that FVIII fused at the N-terminus does not affect a binding site of the Fc variant to FcRn so that the half-life is not affected. In addition, the binding site of protein A in Fc is important for purification in a preparation process. Linking of CTP ensures that FVIII fused at the N-terminus does not “cover” its binding site with protein A so that cheaper and more suitable fillers can be selected to purify the fusion protein, reducing a purification cost. On the other hand, the addition of the CTP enables the Fc fragment of 25 kDa not to interfere with the correct folding of FVIII fused at the N-terminus so that the biological activity/function of FVIII is not decreased or lost. Compared to a flexible linker peptide such as (GGGGS)n that curls irregularly, the rigid CTP polypeptide with multiple glycosyl side chains can form a stable stereo conformation. This “separation” role promotes FVIII and the Fc fragment to be independently folded to form the correct three-dimensional conformation without affecting their respective biological activity. On yet another hand, the protective effect of CTP glycosyl side chains can reduce the susceptibility of the linker peptide to proteases so that the fusion protein is not easily degraded in the linker region.

IgG Fc Variant

Non-Lytic Fc Variant

The Fc element is derived from an Fc fragment in a constant region of immunoglobulin IgG and plays an important role in the immune defense against a pathogen. The effector function of IgG mediated by Fc is performed through the following two mechanisms: (1) Fc binds to Fc receptors (FcγRs) on cell surface, so that the pathogen is digested by phagocytosis or lysis or by killer cells via an antibody-dependent cytotoxicity (ADCC) pathway, or (2) Fc binds to the Clq of a first complement component C1 and triggers a complement-dependent cytotoxicity (CDC) pathway to lyse the pathogen. Among four human IgG subtypes, IgG1 and IgG3 can effectively bind to FcγRs, IgG4 has lower binding affinity to FcγRs, and IgG2 has too low binding affinity to FcγRs to be determined. Therefore, human IgG2 has almost no ADCC effect. In addition, human IgG1 and IgG3 can effectively bind to C1q to activate complement cascade. Human IgG2 weakly binds to C1q and IgG4 does not bind to C1q (Jefferis R et al., Immunol Rev, 1998, 163: 59-76). Therefore, human IgG2 has a weak CDC effect. Apparently, none of the natural IgG subtypes is ideal for constructing a FVIII-Fc fusion protein. To obtain non-lytic Fc with no effector function, the most effective method is to mutate the complement and receptor binding domains of the Fc fragment to regulate the binding affinity of Fc to corresponding receptors, reduce or eliminate ADCC and CDC effects, and retain the biological activity of the functional protein and the extended half-life in vivo of the Fc fragment without producing cytotoxicity. For more mutation sites included in the non-lytic Fc variant, see Shields R L et al., J Biol Chem, 2001, 276(9): 6591-604 or Chinese Patent No. CN 201280031137.2.

Fc Variant with Enhanced Binding Affinity to Neonatal Receptor (FcRn)

The plasma half-life of IgG depends on its binding to FcRn, where IgG generally binds at a pH of 6.0 and dissociates at a pH of 7.4 (the pH of plasma). Through studies on the binding sites, binding sites of IgG to FcRn have been modified to increase the binding capacity at pH 6.0. It has been proved that mutations of some residues of a human Fcγ domain important for the binding to FcRn can increase serum half-life. It has been reported that mutations at T250, M252, S254, T256, V308, E380, M428, and N434 can increase or decrease the binding affinity to FcRn (Roopenian et al., Nat. Rview Immunology, 7: 715-725, 2007). Korean Patent No. KR 10-1027427 discloses trastuzumab (Herceptin, Genentech) variants with enhanced binding affinity to FcRn, where these variants include one or more amino acid modifications selected from 257C, 257M, 257L, 257N, 257Y, 279Q, 279Y, 308F, and 308Y Korean Patent Publication No. KR 2010-0099179 provides bevacizumab (Avastin, Genentech) variants, where these variants exhibit an increased in vivo half-life by containing amino acid modifications at N434S, M252Y/M428L, M252Y/N434S, and M428L/N434S. In addition, Hinton et al. have found that T250Q and M428L mutants increase the binding to FcRn by 3 and 7-fold, respectively. When two site are mutated simultaneously, the binding is increased 28-fold. In rhesus monkeys, M428L or T250QM/428 L mutant exhibits the plasma half-life increased 2-fold (Paul R. Hinton et al., J Immunol, 2006, 176: 346-356). For more mutation sites included in the Fc variant with the enhanced binding affinity to the neonatal receptor (FcRn), see Chinese Patent No. CN201280066663.2. In addition, studies show that T250Q/M428L mutation in the Fc fragment of five humanized antibodies improves the interaction between Fc and FcRn and also show that, in subsequent in vivo pharmacokinetic assays, the Fc mutant antibodies, when administrated by subcutaneous injection, have improved pharmacokinetic parameters such as increased in vivo exposure, reduced clearance, and increased subcutaneous bioavailability compared with a wild-type antibody (Datta-Mannan A et al., MAbs. Taylor&Francis, 2012, 4: 267-273).

Fusion Protein and Preparation Method Thereof

Genes of the fusion protein of the present disclosure are prepared by an artificial synthesis method with optimized codons. According to a nucleotide sequence in the present disclosure, those skilled in the art may conveniently prepare encoding nucleic acids of the present disclosure by various known methods. These methods are not limited to an artificial synthesis or traditional subcloning, etc. For specific methods, see Molecular Cloning: A Laboratory Manual by J. Sambrook. As an embodiment of the present disclosure, an encoding nucleic acid sequence of the present disclosure is constructed by synthesizing segments of the nucleotide sequence and subcloning.

The present disclosure further provides an expression vector of mammal cells, which includes a sequence for encoding the fusion protein of the present disclosure and an expression regulatory sequence operatively linked thereto. The term “operatively linked” or “operably linked” refers to such a case where a certain portion of a linear DNA sequence can regulate or control the activity of other portions of the same linear DNA sequence. For example, if a promoter controls the transcription of a sequence, the promoter is operably linked to the encoding sequence.

The expression vector of mammal cells may use commercially available vectors such as, but not limited to, pcDNA3, pIRES, pDR, pBK, and pSPORT that can be used for the expression in a eukaryotic cell system. Those skilled in the art may also select an appropriate expression vector according to the host cell.

According to the restriction map of the known empty expression vector, those skilled in the art may insert the encoding sequence of the fusion protein of the present disclosure into appropriate restriction sites through restriction enzyme cleavage and splicing according to a conventional method to obtain a recombinant expression vector of the present disclosure.

The present disclosure further provides a host cell that expresses the fusion protein of the present disclosure and contains the encoding sequence of the fusion protein of the present disclosure. The host cells are preferably eukaryotic cells such as, but not limited to, CHO cells, COS cells, 293 cells, and RSF cells.

As a preferred embodiment of the present disclosure, the cells are CHO cells which can preferably express the fusion protein of the present disclosure so that the fusion protein with good activity and stability can be obtained.

The present disclosure further provides a method for preparing the fusion protein of the present disclosure using a recombinant DNA technology, comprising the following steps:

(1) providing a nucleic acid sequence for encoding the fusion protein;

(2) inserting the nucleic acid sequence in (1) into an appropriate expression vector to obtain a recombinant expression vector;

(3) introducing the recombinant expression vector in (2) into an appropriate host cell;

(4) culturing the transfected host cell under conditions suitable for expression; and

(5) collecting a supernatant and purifying a fusion protein product.

The encoding sequence may be introduced into the host cell using various technologies known in the art, such as, but not limited to, calcium phosphate precipitation, lipofection, electroporation, microinjection, viral infection, and alkali metal ions.

For the culture and expression of host cells, see Olander R M et al., Dev Biol Stand, 1996, 86: 338. Cells and residues in a suspension may be removed by centrifugation to collect the supernatant.

The fusion protein prepared above may be purified to obtain substantially homogeneous properties such as a single or particular band on SDS-PAGE electrophoresis. First, the expression supernatant is concentrated and the concentrated solution may be further purified through gel chromatography or ion exchange chromatography such as anion exchange chromatography or cation exchange chromatography. The gel matrix may be a medium commonly used for protein purification, such as agarose, dextran, and polyamide. Q- or SP-groups are relatively ideal ion exchange groups. Finally, the purified product may be further refined by methods such as hydroxyapatite adsorption chromatography, metal chelation chromatography, hydrophobic interaction chromatography, and reversed-phase high-performance liquid chromatography. In all of the above purification steps, different combinations may be used to achieve substantially uniform protein purity. The expressed fusion protein may also be purified using an affinity chromatography column containing an antibody, receptor, or ligand specific for the fusion protein. According to the properties of the affinity column used, fusion polypeptides that bind to the affinity column may be eluted by conventional methods such as a high salt buffer and a pH change.

Pharmaceutical Composition

The present disclosure further provides a pharmaceutical composition containing an effective dose of the fusion protein of the present disclosure and a pharmaceutically acceptable carrier. Generally, an effective amount of the fusion protein of the present disclosure may be formulated in a non-toxic, inert and pharmaceutically acceptable aqueous carrier medium, where the pH is generally about 5-8, preferably, the pH is about 6-8. The term “effective amount” or “effective dose” refers to an amount that can be functional or active to and acceptable by humans and/or animals. “Pharmaceutically acceptable” ingredients are those that are applicable to humans and/or mammals without excessive adverse side effects (such as toxicity, irritation, and allergic reactions), that is, substances with a reasonable benefit/risk ratio. The term “pharmaceutically acceptable carrier” refers to a carrier for the administration of a therapeutic agent, which includes various excipients and diluents.

Pharmaceutically acceptable carriers include (but not limited to): saline, buffer, glucose, water, glycerol, ethanol, and combinations thereof. Generally, a pharmaceutical preparation should match with the mode of administration. The pharmaceutical composition of the present disclosure may be made in the form of injections, for example, prepared by conventional methods using normal saline or aqueous solutions containing glucose and other adjuvants. The pharmaceutical composition is preferably produced under a sterile condition. The amount of active ingredient administered is a therapeutically effective amount. The pharmaceutical preparation of the present disclosure may also be made into a sustained-release preparation.

The effective amount of the fusion protein of the present disclosure may vary depending on the mode of administration and the severity of a disease to be treated. The selection of a preferred effective amount may be determined by those of ordinary skill in the art according to various factors (for example, through clinical trials). The factors include, but are not limited to, the pharmacokinetic parameters of the fusion protein such as bioavailability, metabolism, and the half-life, etc; the severity of the disease to be treated, the weight of a patient, the immune status of the patient, and a path of administration, etc.

### BRIEF DESCRIPTION OF DRAWINGS

FIG. 1 is a diagram of SEC-HPLC spectrogram of a purified fusion protein SS-F8.

FIG. 2 is a diagram of SDS-PAGE electrophoresis of a purified fusion protein SS-F8.

FIG. 3 shows an effect of a fusion protein SS-F8 on bleeding time of saphenous arteries in SD rats.

FIG. 4 shows an effect of a fusion protein SS-F8 on APTT of plasma in SD rats.

FIG. 5 is a statistics diagram of bleeding volume in mice, where *p<0.05, ****p<0.0001.

FIG. 6 is a statistics diagram of bleeding time in mice, where ns p>0.05, ****p<0.0001.

### DETAILED DESCRIPTION

### Example 1. Construction of Expression Plasmids for Encoding Mutant Single-Chain FVIII Fusion Proteins

Gene sequences for encoding an FVIII leader peptide, FVIII protein with a partially deleted B domain, a flexible peptide linker, a CTP rigid unit, and a human IgG vFc variant were artificially optimized codons that CHO cells prefer, and were artificially synthesized. The synthesized full-length DNA fragment of the fusion protein included one restriction endonuclease site, SpeI and EcoRI, at 5′ and 3′ ends, respectively. The full-length DNA fragment was inserted between corresponding restriction sites of pUC57 transfer vector and its sequence was verified through DNA sequencing.

The full-length gene fragment obtained above of the fusion protein was transferred from the intermediate vector to a self-made expression plasmid PXY1A1M between the corresponding restriction sites to obtain a plasmid that expressed high level of the fusion protein. The PXY1A1M plasmid included, but was not limited to, the following important expression elements: (1) a human cytomegalovirus early promoter, and an enhancer required for exogenous high expression in mammal cells; (2) a double screening marker with kanamycin resistance in bacteria and G418 resistance in mammal cells; (3) murine dihydrofolate reductase (DHFR) gene expression cassette. Methotrexate (MTX) can co-amplify fusion genes and DHFR genes when a host cell is deficient in the DHFR genes (see U.S. Pat. No. 4,399,216). The expression plasmid of the fusion protein was transfected into a mammal host cell line. To achieve stable and high-level expression, a preferred host cell line is a DHFR enzyme deficient CHO-cell (see U.S. Pat. No. 4,818,679). After two days of transfection, the medium was replaced with a screening medium containing 0.6 mg/mL G418, and the cells were seeded in a 96-well plate at a certain concentration (5000-10000 viable cells/well) and cultured for 10-14 days until large discrete cell clones appeared. Transfectants resistant to selected drugs were screened through ELISA analysis method. Wells with high-level fusion proteins were subcloned through the extreme dilution of the 96-well culture plate.

A series of mutant single-chain FVIII fusion proteins were constructed in the present disclosure, which contained peptide linkers of different lengths, CTP rigid units of different compositions, and several different subtypes of IgG Fc variants (vFc), to verify the effect of linker peptides and Fc variants on the activity of the mutant single-chain FVIII fusion proteins. Details are shown in Table 1. The amino acid sequences of each component are found in SEQUENCE LISTING.

TABLE 1
Compositions of various single-chain FVIII proteins

| | Compositions of a Series of FVIII Fusion Proteins |
|---|---|
| Code | (from the N-terminus to C-terminus) |
| SS-F4 | scFVIII-L3-CTP¹-vFcγ₁ |
| SS-F5 | scFVIII-L5-CTP⁴-vFcγ₄ |
| SS-F6 | scFVIII-L1-CTP³-CTP³-vFcγ₂₋₂ |
| SS-F7 | scFVIII-L4-CTP³-vFcγ₂₋₁ |
| SS-F8 | scFVIII-L2-CTP²-vFcγ₂₋₃ |
| SS-F9 | scFVIII-L2-vFcγ₂₋₃-CTP² |
| 55-F10 | scFVIII-L1-vFcγ₂₋₃ |
| SS-F11 | scFVIII-L4-vFcγ₂₋₃ |

### Example 2. Screening of a Stably Transfected Cell Line with High Expression of Fusion Protein

The above expression plasmid of a fusion protein was transfected into a mammal host cell line to express the mutant single-chain FVIII fusion protein. To maintain stable and high-level expression, a preferred host cell is a DHFR deficient CHO-cell (see U.S. Pat. No. 4,818,679). A preferred transfection method is electroporation. Other methods may be used, such as calcium phosphate co-precipitation, lipofection, and microinjection. The electroporation method used Gene Pulser Electroporator (Bio-Rad Laboratories) with a voltage of 300 V and a capacitance of 1050 pFd, and 50 pg of Pvul linearized expression plasmids was added to 2 to 3×10⁷ cells placed in a cuvette, and the cells after electroporation were transferred to a shake flask containing 30 mL of growth medium. After two days of transfection, the medium was replaced with a growth medium containing 0.6 mg/mL G418, and the cells were seeded in a 96-well plate at a certain concentration and cultured for 10-12 days until large discrete cell clones appeared. Transfectants resistant to selected drugs were screened through ELISA analysis method against human IgG Fc. Wells with high-level expression of fusion proteins were subcloned through extreme dilution.

To achieve the high-level expression of fusion proteins, DHFR gene suppressed by MTX should be used for co-amplification. The transfected fusion protein genes were co-amplified with the DHFR gene in growth media containing MTX with increased concentrations. Positive subclones that expressed DHFR were subjected to limiting dilution, and the pressure was increased gradually to screen out transfectants that could grow in media with MTX of up to 6 μM. The secretion rates of the transfectants were determined and cell lines with high exogenous protein expression were screened. Cell lines with a secretion rate greater than about 1 (preferably about three) IU/10⁶ (million) cells/24 h were adaptively suspended using a serum-free medium, and the fusion proteins were then purified using a conditioned medium.

### Example 3. Production of Fusion Proteins

The high-yield cell strains preferably obtained in Example 2 were first subjected to serum-free acclimation and culture in a petri dish, and then transferred to a shake flask for suspension acclimation and culture. After the cells were acclimated to these culture conditions, fed-batch culture was performed in a 300 mL shake flask or perfusion culture was simulated by changing media daily. A CHO-derived cell strain that was screened in Example 2 for producing fusion protein SS-F8 was subjected to fed-batch culture for 14 days in a 300 mL shake flask, where a cumulative yield of expressed recombinant fusion proteins reached 200 mg/L and a maximum viable cell density reached 15×10⁶/mL. To obtain more fusion proteins, a 1000 mL shake flask may also be used for culture. In another culture method, the above-mentioned CHO-derived cell strain was cultured in a 100 mL shake flask with media changed daily, where a daily cumulative yield of expressed recombinant fusion proteins was about 20 mg/L and a maximum viable cell density in the shake flask reached 30×10⁶/mL. The biological activity of the recombinant fusion proteins produced by the above two methods was determined to be comparable.

### Example 4. Purification and Qualitization of Fusion Proteins

Fusion protein SS-F8 was mainly purified through four-step chromatography in the present disclosure. The four-step chromatography was respectively affinity chromatography, anion exchange chromatography, hydrophobic chromatography, and molecular sieve chromatography. (the protein purifier used in this example was AKTA pure 25 M from GE, U.S; and reagents used in this example were purchased from Sinopharm Chemical Reagent Co., Ltd and had purity at an analytical grade).

In a first step, affinity chromatography was performed. Sample capture, concentration and the removal of partial pollutants were performed using VIII Select Affinity Chromatography Media from GE. First, the chromatography column was equilibrated with 3-5 column volumes (CVs) of an equilibration buffer containing 10 mM of HEPES, 150 mM of NaCl, 25 mM of CaCl₂), and 0.05% Tween-80 and with a pH of 6.8-7.2 at a linear flow rate of 50-100 cm/h. The clarified fermentation broth was loaded at a linear flow rate of 50-100 cm/h with a load not higher than 50000 IU/mL. After loading, the chromatography column was equilibrated with 3-5 column volumes (CVs) of an equilibration buffer containing 10 mM of HEPES, 150 mM of NaCl, 5 mM of CaCl₂), and 0.05% Tween-80 and with a pH of 6.8-7.2 at a linear flow rate of 50-100 cm/h, and unbound components were rinsed. The chromatography column was rinsed with 3-5 column volumes of a decontamination buffer 1 containing 10 mM of HEPES, 1 M of NaCl, 25 mM of CaCl₂), and 0.05% Tween-80 and with a pH of 6.8-7.2 at a linear flow rate of 50-100 cm/h to remove partial pollutants. The chromatography column was equilibrated with 3-5 column volumes (CVs) of an equilibration buffer containing 10 mM of HEPES, 150 mM of NaCl, 25 mM of CaCl₂), and 0.05% Tween-80 and with a pH of 6.8-7.2 at a linear flow rate of 50-100 cm/h. Then the target product was eluted at a linear flow rate not higher than 50 cm/h using an elution buffer containing 20 mM of His-HCl, 25 mM of CaCl₂), 0.02% Tween-80, and 45% propylene glycol and with a pH of 6.8-7.2 and target peaks were collected.

In a second step, anion exchange chromatography was performed. Intermediate purification was conducted by using Q-HP of Bestchrom or other commercially available anion exchange chromatography media (such as Q HP of GE, Toyopearl GigaCap Q-650 of TOSOH, DEAE Beads 6FF of Smart-Lifesciences, Generik MC-Q of Sepax Technologies, Inc, Fractogel EMD TMAE of Merck, and Q Ceramic HyperD F of Pall) to separate structural variants and further remove pollutants such as HCP and DNA. First, the chromatography column was rinsed with 3-5 column volumes (CVs) of an equilibration buffer containing 20 mM of His-HCl, 100 mM of NaCl, 10 mM of CaCl₂, and 0.02% Tween-80 and with a pH of 7.0-7.5 at a linear flow rate of 50-100 cm/h. The target proteins separated through affinity chromatography in the first step were diluted 5-10 times to reduce the concentration of organics. Then samples were loaded with the load being controlled within 5000-10000 IU/mL. After loading, the chromatography column was rinsed with 3-5 column volumes (CVs) of an equilibration buffer containing 20 mM of His-HCl, 100 mM of NaCl, 10 mM of CaCl₂, and 0.02% Tween-80 and with a pH of 7.0-7.5 at a linear flow rate of 50-100 cm/h. Then, the chromatography column was rinsed with 3-5 column volumes (CVs) of a wash buffer containing 20 mM of His-HCl, 500 mM of NaCl, 10 mM of CaCl₂, and 0.02% Tween-80 and with a pH of 7.0-7.5 at a linear flow rate of 50-100 cm/h to remove partial impurity proteins. The chromatography column was then eluted with gradient salt concentrations using an elution buffer containing 20 mM of His-HCl, 1 M of NaCl, 10 mM of CaCl₂, and 0.02% Tween-80 and with a pH of 7.0-7.5, where the condition was elution buffer from 50% and 60%, 3-5 column volumes (CVs), and a linear flow rate not higher than 50 cm/h. Eluted fractions were collected. The samples were sent for detecting protein content, SEC-HPLC, activity, and HCP content separately. A protein concentration and protein activity were determined.

In a third step, hydrophobic chromatography was performed. Intermediate purification was conducted by using Butyl HP of Bestchrom or other commercially available hydrophobic chromatography media (such as Butyl HP of GE, Toyopearl Butyl-650 of TOSOH, Butyl Beads 4FF of Smart-Lifesciences, Generik MC30-HIC Butyl of Sepax Technologies, Inc, and Fractogel EMD Propyl of Merck) to reduce the content of polymers. The eluate obtained after anion exchange chromatography in the second step still contained a certain proportion of polymers. Because of various reasons for the formation of the polymers such as polymerization with unchanged structures and polymerization with changed structures, the biological activity of the polymers varies greatly and thus causes great interference to the analysis of biological activity. When the target proteins are polymerized, the polymers and monomers differ in property including charge characteristics and hydrophobicity. Polymers and monomers were separated using differences in hydrophobicity. Since the final step of purification would be molecular sieve chromatography, purification was performed using Butyl HP to remove partial polymers to a content less than 10%. First, the chromatography column was equilibrated with 3-5 column volumes (CVs) of an equilibration buffer containing 20 mM of His-HCl, 1.5 M of NaCl, 5 mM of CaCl₂, and 0.02% Tween-80 and with a pH of 6.8-7.2 at a linear flow rate of 50-100 cm/h. The conductivity of the target proteins separated through anion exchange chromatography in the second step was adjusted using a buffer containing 2 M of (NH₄)₂SO₄. Then samples were loaded with a load being controlled lower than 20000 IU/mL. After loading, the chromatography column was rinsed with 3-5 column volumes (CVs) of an equilibration buffer containing 20 mM of His-HCl, 1.5 M of NaCl, 5 mM of CaCl₂), and 0.02% Tween-80 and with a pH of 6.8-7.2 at a linear flow rate of 50-100 cm/h. Then, the chromatography column was rinsed with 3-5 column volumes (CVs) of a wash buffer containing 20 mM of His-HCl, 1.5 M of NaCl, 5 mM of CaCl₂), and 0.02% Tween-80 and with a pH of 6.8-7.2 at a linear flow rate of 50-100 cm/h to remove partial polymers. Finally, the target proteins were eluted respectively with 3-5 column volumes (CVs) of 20%, 40%, and 100% of an elution buffer containing 20 mM of His-HCl, 5 mM of CaCl₂), 0.02% Tween-80, and 50% ethylene glycol and with a pH of 6.8-7.2 at a linear flow rate not higher than 60 cm/h. Eluted fractions were collected and sent for detecting SEC-HPLC separately. Target components with the percentage of monomers being greater than 90% were combined for chromatography in the next step.

In a fourth step, molecular sieve chromatography was performed. Separation was conducted by using superdex 200 of GE or other commercially available molecular sieve media (such as Chromdex 200 prep grade of Bestchrom) to reduce the content of polymers to be lower than 5% and further reduce the content of key pollutans. The chromatography column was rinsed with 2 column volumes (CVs) of an equilibration buffer containing 10 mM of His-HCl, 150 mM of NaCl, 2 mM of CaCl₂), 10 mM of sucrose, and 0.02% Tween-80 and with a pH of 6.8-7.2 at a linear flow rate of 20-40 cm/h. The load was not higher than 3% of the column volume and the samples were rinsed at a linear flow rate of 20 cm/h. Elution components were sequentially collected and combined for SEC detection.

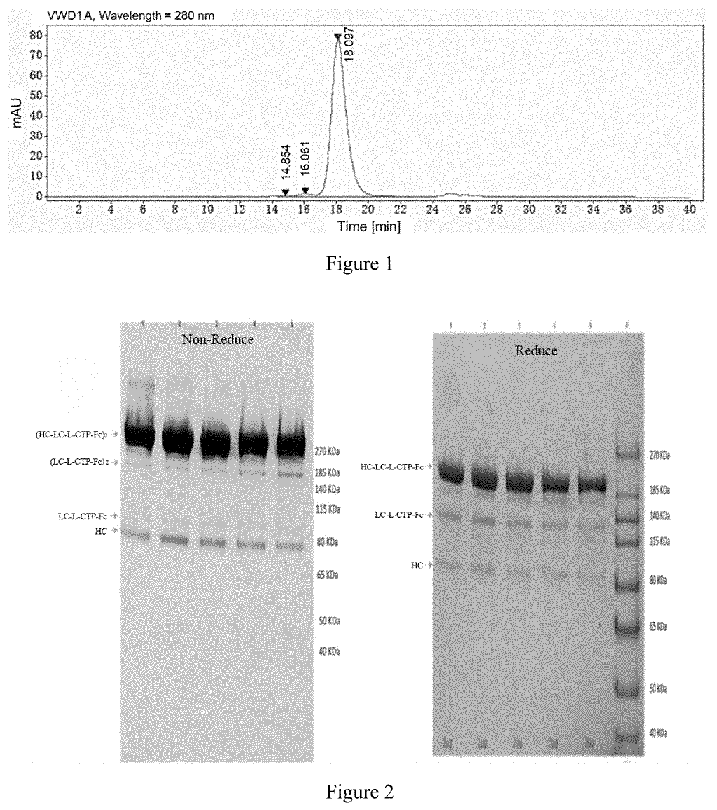

The SEC-HPLC purity result and SDS-PAGE electrophoresis result of the samples are shown in FIG. 1 and FIG. 2 respectively. The SEC-HPLC result shows that the purity of main peaks of the purified fusion proteins was higher than 98.0%. The SDS-PAGE electrophoretic pattern was as expected. The non-reducing electrophoresis included bands of an unprocessed fusion protein (about 390 kDa), a (LC-L-CTP-Fc)₂ dimer fragment (about 210 kDa) formed after shedding of two heavy-chain fragments upon cleavage at another common cleavage site E720, an LC-L-CTP-Fc single-chain fragment (about 100 kDa) and an HC fragment (about 90 kDa). After reduction, clear HC-LC-L-CTP-Fc (about 190 kDa), LC-L-CTP-Fc (about 105 kDa) and HC (about 90 kDa) single-chain bands were obtained. The specific activity of the obtained protein components was 6000-8000 IU/mg.

### Example 5. Indirect Determination of the Activity of the Fusion Protein In Vitro by a Chromogenic Substrate Method

The activity of the mutant single-chain FVIII fusion protein may be determined by the chromogenic substrate method. In this example, the activity was determined using Biophen FVIII:C kit (HYPHEN BioMed, Ref. 221402) based on the following principle: when activated by thrombin, FVIII:C binds to FIXa in the presence of phospholipids and calcium ions to form an enzyme complex which in turn can activate factor X to transform into its active form Xa. The active factor Xa can cause the cleavage of its specific chromogenic substrate (SXa-11) to release chromogenic group pNA. The activity of FXa directly proportional to the amount of pNA may be obtained by determining the amount of produced pNA at 405 nm. In this system, the contents of factor IXa and factor X are certain and excessive, and the activity of FXa is only directly related to the content of FVIIIa. The specific activity of the mutant single-chain FVIII fusion protein determined by this method was about 6000-8000 IU/mg.

### Example 6. Studies on the Stability of the Purified Fusion Protein

To further verify the stability of the mutant single-chain FVIII fusion protein at room temperature of 25° C., the mutant single-chain FVIII fusion protein was stored at room temperature of 25° C. for several days to investigate the effect on the activity of the fusion protein.

A medicine stability testing chamber (purchased from Shanghai Yiheng Scientific Instrument instruments Co., Ltd.) was used to set a test temperature of 25° C. and a humidity of 75%. Eight copies of SS-F8 and double-chain factor VIII reference drug DS-F8 (a fusion protein retaining the protease cleavage site between Arg1648 and Glu1649 of human wild-type FVIII and having an amino acid sequence as shown in SEQ ID NO: 11) that were diluted to the same concentration, 200 μL for each copy, were stored in the medicine stability testing chamber. One copy of SS-F8 and one copy of DS-F8 were taken and the activity of the fusion proteins was assayed according to the method in Example 5 and recorded as activity values on the first day (d1). Then, the activity of the mutant single-chain FVIII fusion protein was assayed separately on d3, d5, d7 and d14 after the fusion proteins were placed at room temperature (temperature: 25° C., humidity: 75%). The results show that the protein activity of SS-F8 only decreased by about 10% and the protein activity of DS-F8 decreased by more than 25% after 5 days at room temperature; the activity of DS-F8 decreased at a significantly higher rate than that of SS-F8 after 7 days at room temperature; and the protein activity of SS-F8 still remained 80% after 7 days. It can be seen that fusion protein SS-F8 has significantly better stability than DS-F8.

### Example 7. Pharmacodynamic Study on the Fusion Protein

Blood coagulation is in fact enzymatic reactions of a series of coagulation factors. The whole coagulation process is divided into three stages: the first stage is the formation of blood thromboplastin; the second stage is the formation of thrombin; and the third stage is the formation of fibrin, where FVIII and FIX are endogenous coagulation factors and FVII is an exogenous coagulation factor. Bleeding time is the time from natural bleeding to natural hemostasis after skin capillaries are punctured. The procoagulant effect of the fusion protein was detected by observing the effect of the mutant single-chain FVIII fusion protein on the bleeding time of saphenous arteries in SD rats.

Seven-week-old SD rats (purchased from Shanghai SLAC Laboratory Animal Co., Ltd) were selected and randomly divided into two groups. The administration group was administered intravenously with SS-F8 at a single dosage of 200 IU/rat and the control group was administered with the same volume of normal saline. Three hours after administration, rats were induced to be anesthetized. After the surgical position was disinfected, the skin was cut from about 1 cm above the medial malleolus through the knee joint to the artery at leg root; subcutaneous tissues were separated from the supra-vascular protective membrane; and venous vessels, arterial vessels and nerves were exposed in sequence. The saphenous artery and its branches were found at the position of the knee joint. Muscle positions within 5 mm*1 mm near the venous vessels were sharp-dissected with microscopic straight forceps, and the veins, arteries and nerves were lifted from the medial side. The saphenous vein and the saphenous artery were cut with a microscissor and the nerves were not touched or injured. The initial bleeding time was the time when the blood gushed out and recorded as t1. The bleeding site was observed every 30 seconds until no bleeding was seen. The time was recorded as hemostasis time t2. The bleeding time was recorded through a calculation of t2-t1. A statistical analysis was performed on experimental data that was expressed as means±a standard deviation (SD). If the data follows normal distribution, SPSS 18.0 software was used for one-way analysis of variance or student's-test. In the case of non-normal distribution, a non-parametric test such as a Kruskal-wallis test or a Mann-whitney test was used, where P≤0.05 denotes a significant difference and P≤0.01 denotes a very significant difference.

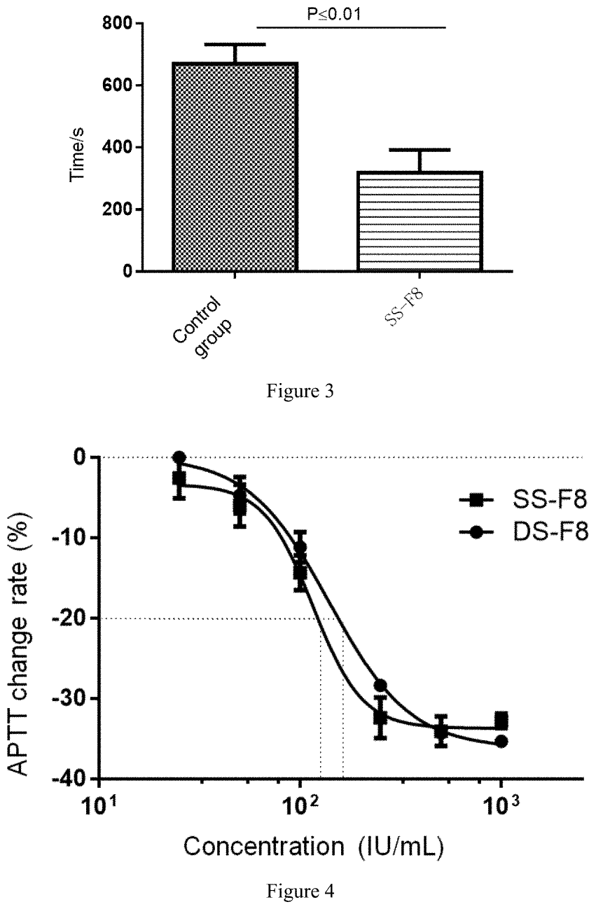

The experimental results are shown in FIG. 3. After prophylaxis administration, the bleeding time of the SS-F8 group was significantly different from that of the normal saline group in statistics after the bleeding of the saphenous artery caused by a surgery in SD rats. At the dosage of 200 IU/rat, the prophylactic administration of SS-F8 has a significant hemostatic effect on the bleeding model.

### Example 8. Direct Determination of the Biological Activity of the Fusion Protein by a Coagulation Method

The in vitro anticoagulant effect of SS-F8 on SD rat plasma was observed by determining activated partial thromboplastin time (APTT).

Seven-week-old SD rats (purchased from Shanghai SLAC Laboratory Animal Co., Ltd) were selected and randomly divided into 12 groups. After induced to be anesthetized, the rats were cut along the median line of the abdomen with a scalpel under continuous anesthesia, and 10-12 mL of blood was taken from the abdominal aorta. The above blood samples were centrifuged at 20° C. and 1500 rpm for 30 min, and supernatant plasma was separated and added to labeled 1.5 mL centrifuge tubes separately. The whole plasma and test drugs SS-F8 and DS-F8 were prepared at a volume ratio of 6:1 into test samples with concentrations of 25-1000 IU/mL respectively. In a control group, a diluent was added at a volume ratio of 6:1. The APTT values of the above samples were tested with a fully automated coagulator (CS-2000i, Sysmex). The change rate of APTT was calculated according to the following formula: the change rate of APTT=(the APTT value of the administration group − the APTT value of the control group)/the APTT value of the control group. The experimental data was expressed as means±a standard deviation (SD). If the data follows normal distribution, SPSS 18.0 software was used for one-way analysis of variance or student's-test. In the case of non-normal distribution, a non-parametric test such as a Kruskal-wallis test or a Mann-whitney test was used, where P s 0.05 denotes a significant difference and P s 0.01 denotes a very significant difference.

It can be seen from FIG. 4 that the test drugs SS-F8 and DS-F8 had anticoagulant effect on the plasma of normal rats; the APTT of the test drugs DS-F8 and SS-F8 at a high concentration of 1000 IU/mL decreased by 33.65% and 31.86%, respectively, compared with the vehicle control group; the EC₅₀ values of DS-F8 and SS-F8 were 139.4 IU/mL and 115.6 IU/mL, respectively; and the EC₅₀ value of SS-F8 was lower than that of DS-F8, which suggests a lower dosage. With an increase in the concentration of the test drugs, the APTT was significantly reduced and there was a dose-effect relationship.

### Example 9. Hemostatic Effect of the Fusion Protein on Acute Hemorrhage in Mice with Hemophilia A

The acute hemostatic effect of SS-F8 in mice with hemophilia A was evaluated through a tail clip bleeding model of VIII factor gene knockout homozygous hemophilia A (HA). Male HA mice at the age of 6-7 weeks (from Shanghai Model Organisms Center, Inc.) were selected, adaptively fed for one week, and randomly divided into two groups: a HA mouse blank vehicle control group and an SS-F8 administration group. Male C57BL/6 mice (purchased from Shanghai SLAC Laboratory Animal Co., Ltd) were selected as a normal control group. Mice were anesthetized through the intraperitoneal injection of 1% sodium pentobarbital (Merck & Co.) at the dosage of 7.5 mL/kg. The C57BL/6 mouse normal control group and the HA mouse blank vehicle control group were administered intravenously via tail veins with a vehicle at the dosage of 10 mL/kg, and the administration group was administered intravenously via tail veins with SS-F8 at the dosage of 112 IU/kg. The tails of the mice were clipped 15 mm from the ends of the tails using a scalpel blade 15 minutes after the administration. The wounds were quickly immersed in 13 mL of normal saline preheated to 37° C. The timing was started, and the blood was collected. The total bleeding time and the bleeding volume within 30 min after the tails were clipped were recorded. Bleeding volume (mL)=(the weight of a centrifuge tube after blood collection (g)−the weight of the centrifuge tube before blood collection (g))/1.0. The experimental groups were compared through T-test testing and analysis software was Graphpad Prism 8.0, where p<0.05 was considered to be statistically significant. Detailed results are shown in Table 2.

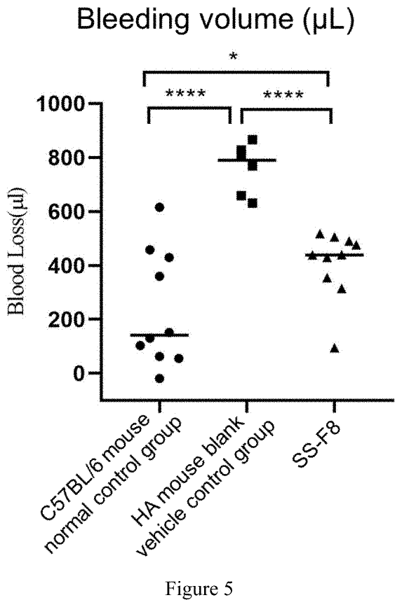

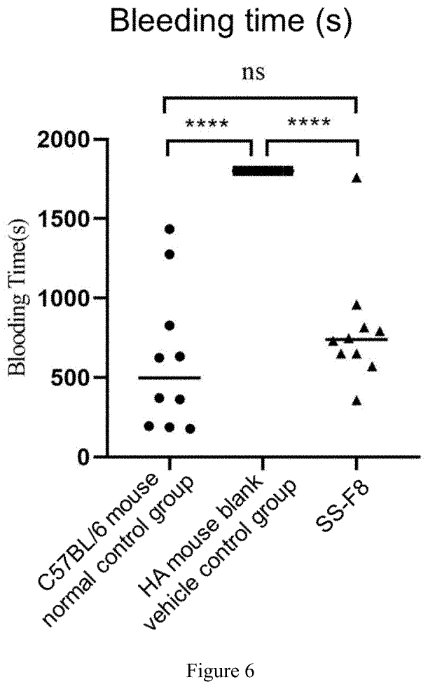

From the analysis of statistical results of the bleeding volume and the sum of bleeding time of each group of animals in FIGS. 5 and 6, the medians of the bleeding volume of the HA mouse blank vehicle control group and the C57BL/6 mouse normal control group were 790.00 μL and 141.15 μL, with an extremely significant difference (P<0.0001), and the medians of the bleeding time were 1800 s and 497 s, with an extremely significant difference (P<0.0001), which indicates that HA mice have an obvious bleeding characteristic compared with normal mice as for the tail-clipping model. After the administration of SS-F8 at the dosage of 112 IU/kg, the medians of the bleeding volume of the administration group and the HA mouse blank vehicle control group were 438.9 μL and 790.00 μL (p<0.0001), and the medians of the bleeding time of the administration group and the HA mouse blank vehicle control group were 739 s and 1800 s (p<0.0001), which indicates a significant coagulant effect and indicates that SS-F8 can be used as an effective coagulant for the acute hemorrhage in hemophilia A, i.e., coagulation factor VIII deficiency.

TABLE 2
Data statistics of the bleeding volume and bleeding time of each group

| | C57BL/6 Mouse | HA Mouse Blank | |
|---|---|---|---|
| | Normal Control | Vehicle Control | SS-F8 Administration |
| Group | Group | Group | Group |
| Bleeding | 141.15 | 790.00 | 438.90 |
| volume (μL) | | | |
| Bleeding time | 497 | 1800 | 739 |
| (s) | | | |

Though the preferred examples of the present disclosure are illustrated and described, it should be understood that those skilled in the art may make various changes in accordance with the teachings herein, and these changes do not violate the scope of the present disclosure.

All the publications mentioned in the present disclosure are incorporated herein by reference as if each publication is separately incorporated herein by reference. In addition, it should be understood that those skilled in the art, who have read the preceding content of the present disclosure, can make various changes or modifications on the present disclosure, and these equivalent forms fall within the scope of the appended claims.

1. A fusion protein of human coagulation factor VIII, comprising sequentially from the N-terminus to the C-terminus a mutant single-chain human coagulation factor VIII with a partially deleted B domain, a flexible peptide linker, at least one carboxyl-terminal peptide rigid unit of human chorionic gonadotropin β-subunit, and a half-life extending moiety; wherein the single-chain human coagulation factor VIII has an amino acid sequence as shown in SEQ ID NO: 2, and the half-life extending moiety is selected from an immunoglobulin Fc fragment, albumin, transferrin, or PEG.

2. The fusion protein according to claim 1, wherein the fusion protein is glycosylated by being expressed in Chinese hamster ovary cells.

3. The fusion protein according to claim 1, wherein the mutant single-chain human coagulation factor VIII with a partially deleted B domain comprises an amino acid sequence as shown in SEQ ID NO: 2, or the single-chain human coagulation factor VIII has an amino acid sequence that shares at least 90% identity to the amino acid sequence as shown in SEQ ID NO: 2.

4. The fusion protein according to claim 1, wherein the flexible peptide linker contains two or more amino acids selected from G, S, A, and T residues;
wherein, the flexible peptide linker has an amino acid sequence general formula formed by combining cyclic unit (GS)a(GGS)b(GGGS)c(GGGGS)d, wherein a, b, c, and d are integers greater than or equal to 0 and a+b+c+d≥1.

5. The fusion protein according to claim 1, wherein the carboxyl-terminal peptide rigid unit of human chorionic gonadotropin β-subunit comprises an amino acid sequence as shown in SEQ ID NO: 3 or a truncated sequence thereof, wherein the truncated sequence comprises at least two glycosylation sites.

6. The fusion protein according to claim 1, wherein the carboxyl-terminal peptide rigid unit of human chorionic gonadotropin β-subunit shares at least 70%, 80%, 90%, or 95% identity to an amino acid sequence as shown in SEQ ID NO: 3, or to a following amino acid sequence:

| (SEQ ID NO: 3) | |
|---|---|
| (i) | PRFQDSSSSKAPPPSLPSPSRLPGPSDTPILPQ; |
| (SEQ ID NO: 12) | |
| (ii) | SSSSKAPPPSLPSPSRLPGPSDTPILPQ; |
| (SEQ ID NO: 13) | |
| (iii) | SSSSKAPPPS; |
| or | |
| (SEQ ID NO: 14) | |
| (iv) | SRLPGPSDTPILPQ. |

7. The fusion protein according to claim 1, wherein the fusion protein comprises 1, 2, 3, 4, or 5 carboxyl-terminal peptide rigid units of human chorionic gonadotropin β-subunit.

8. The fusion protein according to claim 1, wherein the half-life extending moiety is a human IgG Fc variant having a reduced ADCC effect and/or CDC effect and/or enhanced binding affinity with an FcRn receptor.

9. The fusion protein according to claim 1, wherein the fusion protein has an amino acid sequence as shown in SEQ ID NO: 9.

10. The fusion protein according to claim 1, wherein the fusion protein has activity of greater than 6000 IU/mg.

11. DNA for encoding the fusion protein according to claim 1.

12. (canceled)

13. (canceled)

14. A pharmaceutical composition, comprising a pharmaceutically acceptable carrier, excipient, or diluent and an effective dose of the fusion protein according to claim 1.

15. A method for preparing the fusion protein according to claim 1, comprising:
(a) introducing the DNA sequence for encoding the fusion protein into a CHO cell to produce a CHO-derived cell line;
(b) screening in step (a) a high-yielding cell strain that expresses more than 1 IU/10⁶ (million) cells every 24 hours in a growth medium thereof;
(c) culturing the cell strain screened in step (b) to express the fusion protein;
(d) harvesting a fermentation broth obtained in step (c) and separating and purifying the fusion protein.

16. The method according to claim 15, wherein a purification process of the fusion protein in step (d) comprises affinity chromatography, hydrophobic chromatography, anion exchange chromatography, and molecular sieve chromatography.

17. A method for prevention or treatment of a hemorrhagic disease in a patient with a congenital or acquired FVIII deficiency, or spontaneous or surgical bleeding in a patient with hemophilia A, comprising administrating the fusion protein of claim 1 to a patient.

18. The fusion protein according to claim 1, wherein the flexible peptide linker is any one selected from the following group consisting of:

| (SEQ ID NO: 15) | |
|---|---|
| (i) | GSGGGSGGGGSGGGGS; |
| (SEQ ID NO: 16) | |
| (ii) | GSGGGGSGGGGSGGGGSGGGGSGGGGS; |
| (SEQ ID NO: 17) | |
| (iii) | GGGGSGGGGSGGGGSGGGGS; |
| (SEQ ID NO: 18) | |
| (iv) | GSGGGGSGGGGSGGGGSGGGGSGGGGSGGGGSGGGGS; |
| and | |
| (SEQ ID NO: 19) | |
| (v) | GGGSGGGSGGGSGGGSGGGS. |

19. The fusion protein according to claim 1, wherein the carboxyl-terminal peptide rigid unit of human chorionic gonadotropin β-subunit comprises an amino acid sequence selected from the following group consisting of:

| (SEQ ID NO: 13) | |
|---|---|
| (i) | PRFQDSSSSKAPPPSLPSPSRLPGPSDTPILPQ; |
| (SEQ ID NO: 12) | |
| (ii) | SSSSKAPPPSLPSPSRLPGPSDTPILPQ; |
| (SEQ ID NO: 13) | |
| (iii) | SSSSKAPPPS; |
| and | |
| (SEQ ID NO: 14) | |
| (iv) | SRLPGPSDTPILPQ. |

20. The fusion protein according to claim 8, wherein the Fc variant is any one selected from the following group consisting of:
(i) human IgG1 hinge region, CH2 region, and CH3 region containing Leu234Val, Leu235Ala, and Pro331 Ser mutations;
(ii) human IgG2 hinge region, CH2 region, and CH3 region containing Pro331 Ser mutation;
(iii) human IgG2 hinge region, CH2 region, and CH3 region containing Thr250Gln and Met428Leu mutations;
(iv) human IgG2 hinge region, CH2 region, and CH3 region containing Pro331Ser, Thr250Gln, and Met428Leu mutations; and
(v) human IgG4 hinge region, CH2 region, and CH3 region containing Ser228Pro and Leu235Ala mutations.

21. The DNA according to claim 11, which has a sequence as shown in SEQ ID NO: 10.
